(19) United States
(12) Patent Application Publication
(10) Pub. No.: US 2008/0071284 A1
(43) Pub. Date: Mar. 20, 2008

(54) Device for Manipulating and Supplying Hollow or Intervertebral or Disk Prosthesis With Flowable Osteocementum

(76) Inventors: Beat Lechmann, Grenchen (CH); Robert Frigg, Bettlach (CH); Roger Buerki, Balsthal (CH)

Correspondence Address: STROOCK & STROOCK & LAVAN, LLP, 180 MAIDEN LANE, NEW YORK, NY, 10038, US

(21) Appl. No.: 11/587,722

(22) PCT Filed: Apr. 26, 2004
(86) PCT No.: PCT/CH04/00251

§ 371 Date: Aug. 13, 2007

(51) Int. Cl. A61B 17/58

(52) U.S. Cl. 606/99

(57) ABSTRACT

A manipulation device of hollow intervertebral or disk prostheses and to the supply thereof with a flowable, hydraulic, osteocementum. Said device comprises a tube provided with a front end and a rear end, a handle and a guiding element for the flowable osteocementum. The front end of the tube is embodied in such a manner that the handle or the guiding element for the flowable osteocementum can be coupled thereto. The rear end of the tube is embodied in such a manner that it can be connected in a rotationally fixed and sealed manner to a hollow intervertebral or disk prosthesis. The handle can be connected in a rotationally fixed manner to the tube and the tube is torsionally fixed in relation to the longitudinal axis thereof.

### FIELD OF THE INVENTION

The invention relates to an intervertebral prosthesis or disk prosthesis, especially for arthrodesis surgery by means of dorsal access PLIF (posterior lumbar interbody fusion), TLIF (transforaminal lumbar interbody fusion), ELIF (extraforaminal lumbar interbody fusion), ALIF (anterior lumbar interbody fusion) and ACIF (anterior cervical interbody fusion. The objective of this surgical technique is the treatment of a degenerated or otherwise diseased intervertebral disk. The surgeon looks for access to the intervertebral disk through a centrally placed skin incision. Subsequently, he exposes the rear region of the movement segments, especially the laminae and the pedicle entry points. By means of a partial resection of the facettal and laminar components, the surgeon aims past the nerve roots and the medullary space in the direction of the diseased intervertebral disk.

### BACKGROUND OF THE INVENTION

For this surgical technique, only a limited amount of autologous spongiosa is available for filling the cavities of cage-like intervertebral or disk prosthesis and the spaces between individual implants and their surroundings. In the long term, the arthrodesis takes place not with the implant but between the bone and the bone replacement material. The individual implants therefore function only as place holders or spacers.

The intervertebral spaces, supplied with the known intervertebral implants, therefore frequently do not attain complete arthrodesis, that is, they end in a pseudoarthrosis. The situation is much the same also with cage-like intervertebral implants for the cervical spine, as well as for those, which were inserted through ventral entrances. Such intervertebral spaces are not stable mechanically, as would have been expected from a stiffening. The consequences then may be recurring pain with subsequent revision surgery.

For the implants and surgical techniques described above, the surgeon uses autologous bone material, which he obtains from the resected parts of the vertebral body or by means of an additional intervention in the crest of the ilium. Since dorsal accesses to the intervertebral disk space are very narrow, the applying of bone material is made difficult. The surgeon is unable to ensure that the whole of the intervertebral space is filled with autologous bone material. There is therefore the danger that empty spaces will result which, on the one hand, permits migration of the implant. On the other hand, the spaces, not filled with autologous bone material, are filled by a soft, fibrous tissue.

In the case of the known devices for manipulating and supplying hollow intervertebral or disk prostheses with flowable osteocementum, an injection device for the osteocementum must subsequently be brought into the already implanted prosthesis. As is well known, however, the manipulation through the dorsal incision is associated with risks, since access to the intervertebral space passes by the nerve roots and the spinal marrow. It is therefore disadvantageous if, after removal of the implant holder, an injection device must subsequently still be fastened to the implant. The manipulation of such an injection device, which must be introduced, may damage neural structures.

### SUMMARY OF THE INVENTION

The invention is to provide a remedy here. It is an object of the invention to provide a device for manipulating and supplying hollow intervertebral or disk prostheses with flowable osteocementum, which permits a liquid and curable osteocementum (synthetic bone replacement) to be injected and, moreover, after the intervertebral implants (for example, in the form of cages) have been implanted in the intervertebral space.

Pursuant to the invention, this objective is accomplished with a device for manipulating and supplying hollow intervertebral or disk prostheses with flowable osteocementum, which has the distinguishing features of claim 1, as well as with a method for manipulating and supplying hollow intervertebral or disk prostheses, having perforations, with flowable osteocementum, which has the distinguishing features of claim 7.

For this purpose, the inventive device is constructed in several parts. In the first phase of the implantation method, it serves as a holder for the implant and, in the second phase, it serves as an injection aid for the osteocementum.

The advantages achieved by the invention are the following:
a) The connection of the tube with the hollow intervertebral or disk prosthesis remains until the osteocementum has cured and, with that, the intervertebral implant is anchored definitively, so that the manipulation with the injection device for the osteocementum always takes place outside of the wound, that is, at a distance from the neural structures. The length of the tube of the inventive device is such (typically between 5 and 25 cm) that, on the one hand, the manipulation with the osteocementum syringe can take place outside of the wound and, on the other, the flow path for the osteocementum, which has not yet cured, is shortened. The distinctive length is then appropriate if the surgeon uses the retractors in addition, and, for this reason, must overcome a larger distance between the site and the region of his hands. If the osteocementum must flow over an excessively long path (very long tube plus handle), the danger exists that the osteocementum will cure already before it enters the intervertebral space. With that, the filling of the intervertebral space with osteocementum would no longer be ensured;
b) the implant is secured by the emergence and subsequent curing of the flowable hydraulic osteocementum in the intervertebral space;
c) the injection aid (tube) can be connected temporarily firmly with the implant. This makes a safe manipulation of the implant possible. However, the tube can also be connected liquid-tight with the implant, so that the flowable osteocementum can be brought into the intervertebral space without leakage to the neural structures.
d) The multi-part construction of the inventive device has the further advantage that the flow path for the flowable osteocementum is shortened, that is, the inventive device takes the rheological properties of the osteocementum into consideration and
e) the part of the device, which comes into contact with the osteocementum and, after the curing, can no longer be cleaned, are reduced to a minimum.

For a special embodiment, the conveying unit for the flowable osteocementum is constructed as an osteocementum syringe with injection cannulae or as a conveyor screw.

For a further embodiment, the handle is mounted in one piece at the front end of the tube. Preferably, the rear end of the tube has a terminal expansion, which permits an axial rotation of the intervertebral prosthesis about the longitudinal axis of the tube. The expansion may be a “Nut-Verbindung zum Zug” (matched joint to the control wire). In addition, the expansion may be centrally drilled.

In the case of a special embodiment of the inventive method, for which a tube with a handle, attached in one piece thereto, is used, step B may be eliminated. For a different embodiment of the method, the handle is removed from the tube after step C has been carried out.

### BRIEF DESCRIPTION OF THE DRAWINGS

The invention and further developments of the invention are described in even greater detail in the following by means of several examples and partially diagrammatic drawings, in which

FIG. 1 shows a perspective view of a hollow, perforated intervertebral prosthesis, to which a tube has been connected tightly,

FIG. 2 shows a perspective view of the device of FIG. 1 with a handle coupled to the front end of the tube,

FIG. 3 shows a perspective view of the device of FIG. 1 with a conveying unit for the flowable osteocementum coupled to the front end of the tube, before the unit is actuated,

FIG. 4 shows a perspective view of the device of FIG. 3 after the conveying unit is actuated, with the osteocementum emerging from the perforation of the intervertebral prosthesis,

FIG. 5 shows a perspective view of a hollow, perforated intervertebral prosthesis, to which a tube, which is provided in one piece with a handle, is connected tightly,

FIG. 6 shows a perspective view of the device of FIG. 5 with a conveying unit for flowable osteocementum coupled to the front end of the tube, before the unit is actuated and

FIG. 7 shows an enlarged partial view of FIG. 4 in the region of the intervertebral implant.

### DETAILED DESCRIPTION OF THE PREFERRED EMBODIMENTS

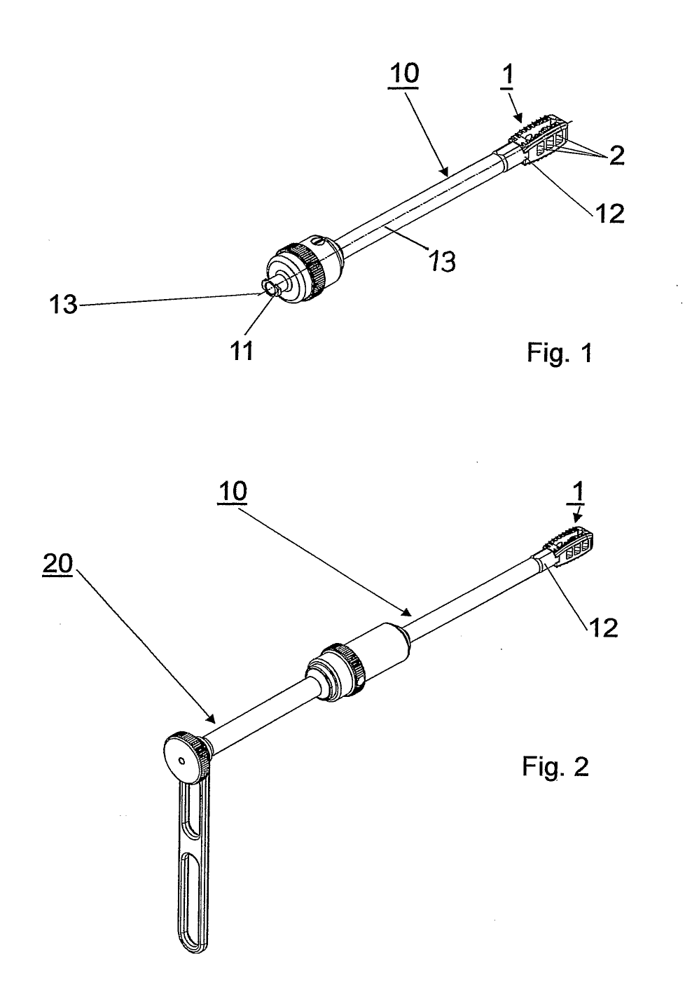

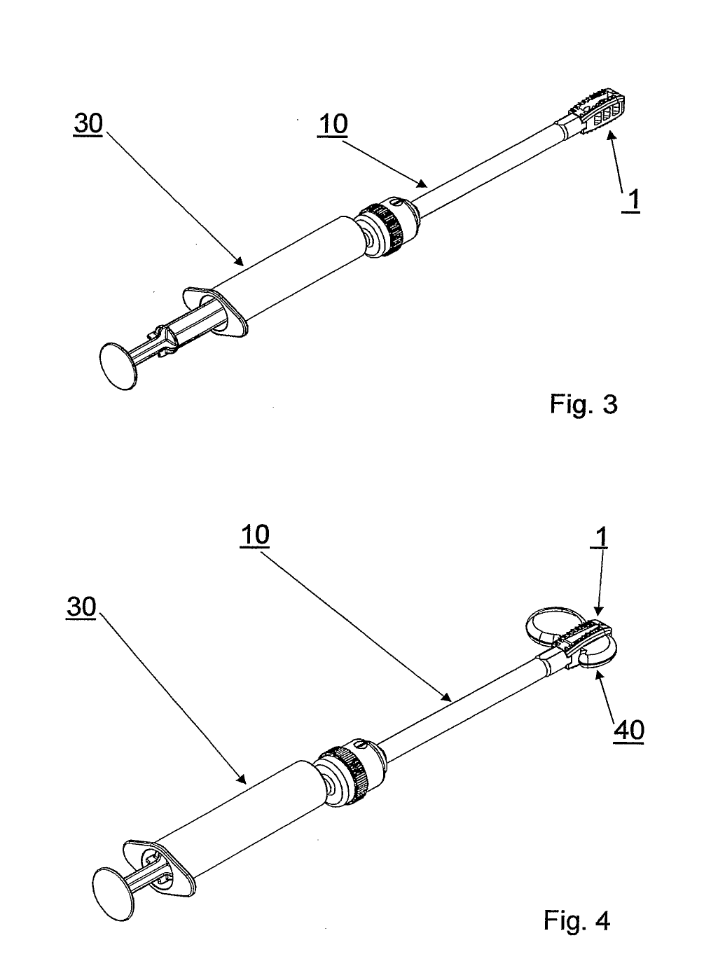

The device for manipulating and supplying hollow intervertebral and disk prostheses 1 with a flowable hydraulic osteocementum, shown in FIGS. 1-4, comprises a tube 10 with a front end 11 and a rear end 12, the latter being connected tightly to a hollow, perforated intervertebral prosthesis 1. A handle 20 is coupled to the front end 11 of the tube 10, as shown in FIG. 2. With this handle 20, it is possible to manipulate the intervertebral prosthesis 1 over the tube 10, which is fastened to the intervertebral prosthesis 1. After the intervertebral prosthesis 1 has been positioned in the intervertebral space, the handle 20 is removed and, as shown in FIG. 3, replaced by a conveying unit for the flowable osteocementum 30. The conveying unit for flowable osteocementum 30 may consist of an osteocementum syringe, which is customarily used for this purpose.

The front end 11 of the tube 10 is constructed in such a manner that, alternatively, the handle 20 or the osteocementum syringe 30 can be coupled to it.

The rear end 12 of the tube 10 is constructed in such a way, that it can be connected rotationally fixed and tightly with the hollow intervertebral or disk prosthesis 1. The handle 20 can also be connected rotationally fixed with the tube 10 and, moreover, the tube 10 is torsionally fixed with respect to its longitudinal axis 13.

As shown in FIG. 4, liquid osteocementum 40 emerges from the perforations 2 of the intervertebral prosthesis 1 after the conveying unit is actuated and solidifies in the specified curing time, as a result of which the intervertebral prosthesis 1 is “cemented” into the intervertebral space.

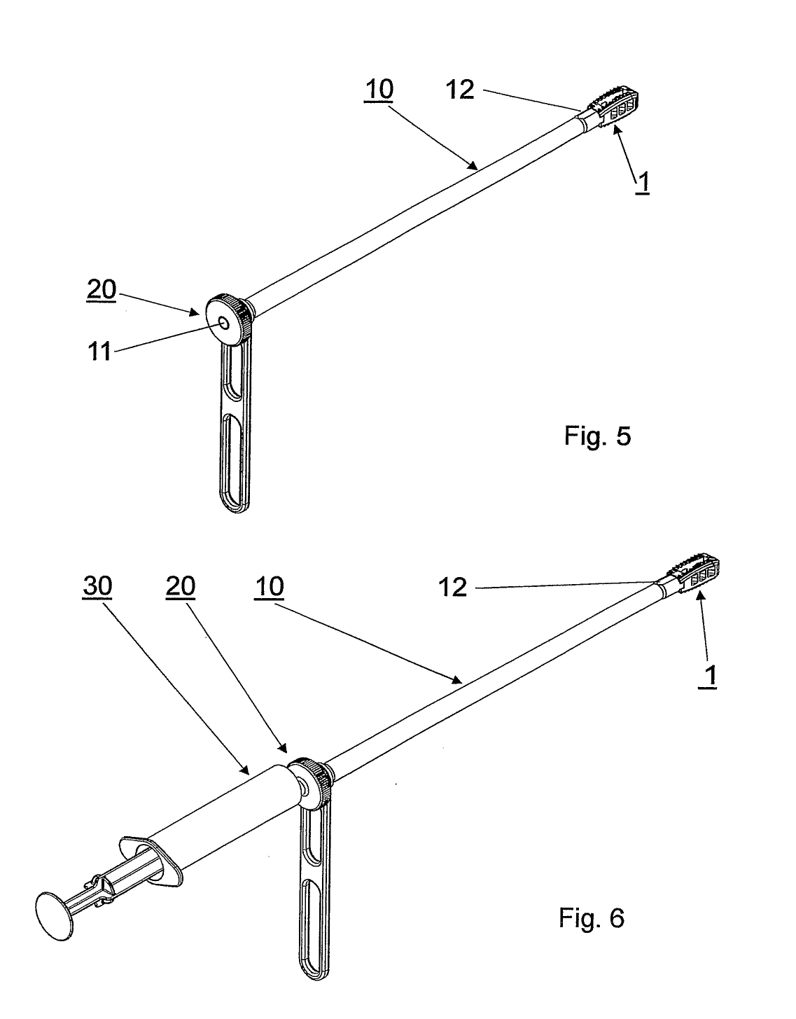

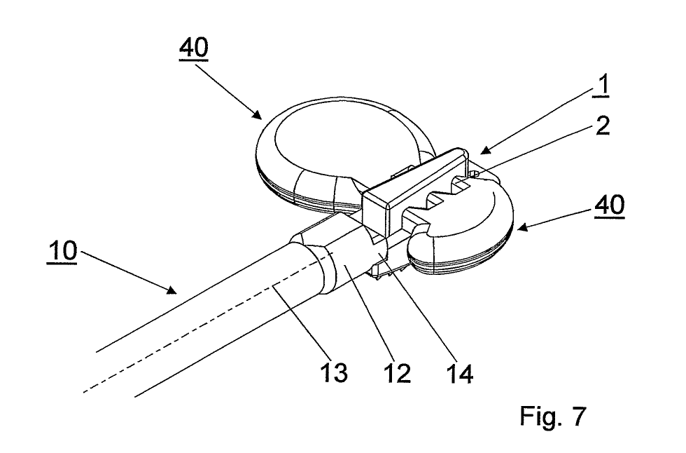

A variation of the inventive device for manipulating and supplying hollow intervertebral or disk prostheses 1 with a liquid hydraulic osteocementum is shown in FIGS. 5-7. For this variation, the tube 10 is connected in one piece with the handle 20. As in the case of the construction FIGS. 1-4, the tube 10 is connected at the rear end 12 with a hollow, perforated intervertebral prosthesis 1. As shown in FIG. 6, the conveying unit for the flowable osteocementum 30 can be coupled in the form of an osteocementum syringe to the front end 11 of the tube 10 directly at the handle 20. FIG. 7 shows how the osteocementum 40 emerges from the perforations 2 of the intervertebral prosthesis 1 after the osteocementum syringe is actuated.

The terminal expansion 14 at the tube 10 permits axial rotation of the intervertebral prosthesis 1 about the longitudinal axis 13 of the tube 10. A matched joint for the control wire is suitable for this purpose. This connection is also drilled centrally as is the connection at the implant, so that the osteocementum 40 can flow well through the expansion 14.

1. (canceled)

2. (canceled)

3. (canceled)

4. (canceled)

5. (canceled)

6. (canceled)

7. (canceled)

8. (canceled)

9. (canceled)

10. A device for inserting an intervertebral implant between adjacent vertebra and for supplying the implant with a liquid flowable osteocementum, the device comprising:
a tube having a first end, a second end and a longitudinal axis, the second end of the tube is sized and configured to engage the implant;
a handle operatively associated with the first end; and
a conveying unit containing the liquid flowable osteocementum, the conveying unit being sized and configured to engage the first end;
wherein
the handle is sized and configured to be rotationally fixed with respect to the tube.

11. The device of claim 10, wherein the conveying unit for the flowable osteocementum is an osteocementum syringe.

12. The device of claim 11, wherein the osteocementum syringe includes a conveyor screw.

13. The device of claim 10, wherein the handle is integrally formed with the tube.

14. The device of claim 13, wherein the conveying unit engages the handle.

15. The device of claim 14, wherein the implant is permitted to rotate about the longitudinal axis of the tube.

16. The device of claim 10, wherein the engagement between the implant and the tube is liquid tight.

17. The device of claim 10, wherein the tube engages the implant so that the implant is rotationally fixed with respect to the tube.

18. The device of claim 10, wherein the tube is torisonally fixed with respect to the longitudinal axis of the tube.

19. The device of claim 10, wherein the implant includes a hollow cavity and a plurality of openings configured such that the ostecocementum flows from the conveying unit, through the tube, into the implant, out the plurality of openings and into a space between the vertebra.

20. A device for inserting an intervertebral implant between an upper vertebra and a lower vertebra, the device comprising:
a tube having a first end, a second end and a center bore extending from the first end to the second end, the first end being sized and configured to releasably engage the implant;
a conveying unit containing a flowable osteocementum, the conveying unit being sized and configured to releasably engage the tube such that, when the conveying unit engages the tube, the osteocementum flows from the conveying unit, through the center bore of the tube and into the implant.

21. The device of claim 20, wherein the conveying unit releasably engages the first end of the tube.

22. The device of claim 20, wherein the implant includes a hollow cavity and a plurality of openings so that the ostecocementum flows out of the plurality of openings and into a space between the upper and lower vertebra.

23. The device of claim 20, wherein the conveying unit is a syringe.

24. The device of claim 20, further comprising a handle operatively associated with the tube.

25. The device of claim 24, wherein the handle is integrally formed with the tube.

26. The device of claim 25, wherein the conveying unit engages the handle.

27. The device of claim 26, wherein the implant is permitted to rotate about the longitudinal axis of the tube.

28. The device of claim 20, wherein the second end of the tube has a terminal expansion, which permits axial rotation of the implant about a longitudinal axis of the tube.

29. The device of claim 20, wherein the engagement between the implant and the tube is liquid tight.

30. A method for inserting an intervertebral implant between adjacent vertebra, the method comprising the following steps:
A) coupling the first end of a tube having a central bore to the invertebral implant;
B) inserting the implant between the adjacent vertebra;
C) coupling a conveying unit containing a liquid flowable osteocementum;
D) injecting the flowable osteocementum through the tube, through the implant,and into a space between the adjacent vertebra.
E) disconnecting the tube from the implant.

31. The method of claim 30, further comprising coupling a handle to the tube.

32. The method of claim 31, wherein the handle is removed from the tube after step B.
